(19) United States
(12) Patent Application Publication
(10) Pub. No.: US 2022/0035900 A1
(43) Pub. Date: Feb. 3, 2022

(54) Enhanced Authentication for IMD Communication

(71) Applicant: BIOTRONIK SE & Co. KG, Berlin (DE)

(72) Inventors: Dawn Gayle Flakne, Portland, OR (US); Benjamin Edward Stickrod, Sherwood, OR (US)

(73) Assignee: BIOTRONIK SE & Co. KG, Berlin (DE)

(21) Appl. No.: 17/299,167

(22) PCT Filed: Nov. 13, 2019
(86) PCT No.: PCT/EP2019/081220

§ 371 Date: Jun. 2, 2021

Related U.S. Application Data
(60) Provisional application No. 62/778,314, filed on Dec. 12, 2018.

(51) Int. Cl. G06F 21/32; A61N 1/372; G16H 40/67; G06F 21/35

(52) U.S. Cl. CPC G06F 21/32; A61N 1/37223; G06F 21/35; G16H 40/67; A61N 1/37247

(57) ABSTRACT

The present invention relates to a method for establishing an access of an external device to an implantable medical device, comprising the steps of: Allowing the implantable medical device to assume an activated mode by letting a user of the implantable medical device apply a magnetic field to the implantable medical device, wherein in the activated mode the implantable medical device is enabled to receive authentication information for authenticating the user of the implantable medical device, and providing authentication information to the implantable medical device, when the latter is in the activated mode to establish said access. Furthermore, the present invention relates to a corresponding medical system.

### CROSS-REFERENCE TO RELATED APPLICATIONS

This application is the United States national phase under 35 U.S.C. § 371 of PCT International Patent Application No. PCT/EP2019/081220, filed on Nov. 13, 2019, which claims the benefit of U.S. Patent Application No. 62/778,314, filed on Dec. 12, 2018, the disclosures of which are hereby incorporated by reference herein in their entireties.

### TECHNICAL FIELD

The present invention relates to a method for establishing an access of an external device to an implantable medical device.

### BACKGROUND

Secure communications between an external device (e.g. a programming and/or data display device) and an implantable medical device (IMD) is important to ensure that the person using the external device is known and/or authorized by the patient.

During secure communications between an external device and an implantable medical device (IMD) it is important to ensure that only authorized actors are allowed to communicate with the implantable medical device, particularly when the latter is implanted in a patient. Unauthorized actors may attempt to steal information or change/deny therapy. By utilizing multiple factors, one or more of which is specific to and/or is known only by the patient, communication can be limited to only users who are authorized by the patient.

One particular solution is to require a proximity based mechanism to trigger the initiation of communications between the external device and an IMD.

Furthermore, U.S. Pat. No. 9,596,224 discloses a method of communicating with an implantable medical device, wherein an authentication process is performed to verify an identity of a user of a mobile computing device. A request is received from the user to access an implantable medical device via the mobile computing device. Based on the identity of the user, a first user interface suitable for the user is selected from a plurality of user interfaces that are each configured to control an implantable medical device. The plurality of user interfaces has different visual characteristics and different levels of access to the implantable medical device. The first user interface is displayed on the mobile computing device.

However, any single authentication mechanism has weaknesses that could be exploited to allow an unauthorized actor to obtain data from and send program data to an IMD. Using multi factor authentication strengthens security by providing layers of protection, each factor compensating for potential weakness(es) in other factors.

The present disclosure is directed toward overcoming one or more of the above-mentioned problems, though not necessarily limited to embodiments that do.

### SUMMARY

It is therefore an objective of the present invention to provide a method and a system that are improved regarding security.

To at least this end, a method for establishing an access of an external device to an implantable medical device is disclosed, comprising the steps of:
Allowing the implantable medical device to assume an activated mode by letting a user of the implantable medical device apply a near field signal to the implantable medical device, wherein in the activated mode the implantable medical device is enabled to receive authentication information for authenticating the user of the implantable medical device, and
Providing authentication information to the implantable medical device, when the latter is in the activated mode to establish said access.

Particularly, the user is a patient carrying the IMD which is implanted in the patient.

Particularly, in the activated mode, the IMD prompts the user to input said authentication information. According to an embodiment, the IMD can be configured to prompt the user to input the information through the external device.

Preferably, according to an embodiment of the present invention, said near field signal is applied by placing a near field communication device in proximity to the implantable medical device. According to an embodiment, the near field communication device is a magnet.

According to a further embodiment, the method further comprises the step of allowing the external device to control the implantable medical device when the external device has access to the implantable medical device, wherein particularly the external device is configured to control the IMD by transmitting programming data and/or programming commands to the IMD.

According to a further embodiment of the method, said authentication information comprises biometric data of the user.

Particularly, in an embodiment, said biometric data is one of: a heart rate of the user, a heart interval pattern of the user, a temperature of the user, a retina pattern of the user, a fingerprint of the user, a respiration rate of the user, a knuckle pattern of the user.

Particularly, according to an embodiment, providing said authentication information involves measuring biometric data of the user by means of the IMD as well as by means of the external device, and transmitting the measured biometric data measured by the external device from the external device to the IMD. Particularly, in an embodiment, the method comprises the further step of permitting access of the external device to the implantable medical device if the transmitted biometric data matches the biometric data measured by the IMD. Particularly, the biometric data can be a series of heart intervals of the patient. Other biometric data of the patient (e.g. as disclosed herein) can also be used.

Furthermore, according to an embodiment, providing said authentication information involves requesting the user (e.g. through the external device) to modify a respiration rate of the user (e.g. take three slow breaths) and measuring the respiration rate of the user by means of the IMD. Particularly, in an embodiment, the method comprises the further step of permitting access of the external device to the implantable medical device if the measured respiration rate matches the requested modification.

Furthermore, according to an embodiment, providing said authentication information to establish said access involves inputting authentication information by the user (e.g. via the external device), e.g. by machine-reading (e.g. scanning) of authentication information (e.g. a barcode) by the user, which authentication information has been stored in the IMD before, particularly during manufacturing of the IMD, particularly to verify that the user (e.g. a patient carrying the IMD implanted in the patient) is the one initiating access to the IMD. Particularly, the authentication information can be kept by the manufacturer and/or can be retrievable by the user. Particularly, in an embodiment, the method comprises the further step of permitting access of the external device to the implantable medical device if the authentication information input by the user corresponds to the authentication information stored in the implantable medical device.

Furthermore, according to an embodiment, providing said authentication information involves inputting authentication information by the user (e.g. via the external device), wherein particularly said authentication information (e.g. one or several of: name, date of birth, address, Physician's Name, password, PIN) has been programmed into the IMD after implantation by means of a privileged external device (e.g. a programmer). Normally, these fields are not writable by a patient remote type device. During the security exchange, the authentication information (or a hash) can be provided via the external device to establish access to the IMD.

Particularly, according to an embodiment, providing said authentication information involves inputting of a password by the user via the external device (e.g. a patient carrying the IMD implanted in the patient). Particularly, in an embodiment, the method comprises a further step of permitting access of the external device to the implantable medical device if the password input by the user matches a password stored in the IMD.

Furthermore, according to an embodiment, before said inputting of said password, the method comprises the further step of creating the password by the user and storing the password in the IMD after implantation of the IMD (e.g. while visiting a clinician after implantation).

Further, in an embodiment, the password is stored in the IMD by a clinician upon adjusting and/or assigning the IMD to the user (e.g. the clinician may use a device with elevated privileges).

Further, in an embodiment, after adjusting and/or assigning the IMD to the user, said step of allowing the implantable medical device to assume the activated mode is conducted by applying a near field to the implantable medical device.

Further, in an embodiment, the method comprises the further step of establishing an encrypted connection between the external device and the IMD.

Further, in an embodiment, the method comprises the further step of letting the external device prompt the user to input the password that had been previously stored in the IMD.

Further, in an embodiment, the method comprises the further step of transmitting a representation of the password via the encrypted connection to the IMD.

Furthermore, according to an embodiment, the method comprises the further step of letting the IMD decrypt the transmitted representation of the password and compare the transmitted password representation with the password representation stored in the IMD.

Particularly, in an embodiment, the method comprises the further step of permitting access to the IMD if the representation of the password input by the user matches a password representation stored in the IMD, and allowing the external device to control the IMD.

Furthermore, according to yet another embodiment, providing said authentication information involves prompting the user (e.g. a patient carrying the IMD implanted in the patient) to move according to a pre-defined movement pattern (e.g. the external device could prompt the patient to tap the IMD with a defined pattern or to sit still for a pre-defined amount of time or to move while initiating communication), and detecting said movement pattern with an accelerometer comprised by the IMD. Particularly, in an embodiment, the method comprises the further step of permitting access of the external device to the implantable medical device if the detected pattern matches the pre-defined movement pattern. According to an example, the external device prompts the user to tap the IMD a plurality of times (e.g. five times) with a pre-defined pause (e.g. one second) in between each two successive taps. Alternatively, the external device can prompt the user to sit motionless for a pre-defined amount of time (e.g. 10 seconds).

Furthermore, according to an embodiment, providing said authentication information involves prompting the user (e.g. a patient carrying the IMD implanted in the patient) by the external device to place a hand over the IMD, and detecting the presence of the hand by capacitive sensing performed by the IMD. Particularly, in an embodiment, the method comprises the further step of permitting access of the external device to the implantable medical device, if a detection signal generated by the IMD matches a pre-defined reference confirming said presence of the hand over the IMD.

Alternatively, providing said authentication information involves prompting the user (e.g. a patient carrying the IMD implanted in the patient) by the external device to press against the IMD, and detecting deformation of the IMD due to said pressing by means of a strain gauge of the IMD. Particularly, in an embodiment, the method comprises the further step of permitting access of the external device to the implantable medical device, if a detection signal generated by the strain gauge matches a pre-defined reference confirming said pressing against the IMD.

Furthermore, according to an embodiment, providing said authentication information to establish said access involves prompting the user (e.g. a patient carrying the IMD implanted in the patient) to press a button on the external device or to apply a magnetic field to the IMD for a second time.

According to an embodiment of the present invention, the external device may communicate with the IMD via radio frequency (RF) communication using a communication coil/antenna. For the communication, e.g. Bluetooth Low Energy (BLE) or the MICS (Medical Implant Communication Service) frequency band is used which is commonly applied for transmissions for monitoring of medical implants. Moreover, high energy pulses can be applied for the authentication or the communication process between external device and IMD. High energy pulses can be used also as trigger signal for announcing an upcoming data transmission from/to the IMD or the external device, or as wakeup signal for converting the IMD and/or the external device from a dormant state into an active state.

Further, in an embodiment, providing said authentication information to establish said access comprises applying a charging device to the IMD to charge a battery of the IMD. Particularly, in an embodiment, the method comprises the further step of permitting access of the external device to the implantable medical device if the battery is being charged by the charging device.

Furthermore, in an embodiment, providing said authentication information to establish said access comprises emitting a light pattern (e.g. by means of the external device or some other device), and detecting said light pattern by means of a light sensor of the IMD. Particularly, in an embodiment, the method comprises the further step of permitting access of the external device to the implantable medical device if the detected light pattern corresponds to a pre-defined reference.

In each of the above-described embodiments, access of the external device to the IMD may only be permitted if in addition one or several further authentication procedures have also been completed successfully.

A further aspect of the present invention relates to a medical system that is configured to establish an access of an external device to an implantable medical device, wherein the medical system comprises:
an implantable medical device,
an external device configured to control the implantable medical device when the external device has access to the implantable medical device,
a device capable of generating a near field signal, such as a magnet, configured to be manually positioned by a user of the implantable medical device for applying a near field signal to the implantable medical device (particularly when the device is positioned in proximity to the implantable medical device), wherein the implantable medical device is configured to assume an activated mode when the near field signal is applied to the implantable medical device by the device, and wherein in the activated mode the implantable medical device is configured to receive authentication information (e.g. a security key) related to the user, and wherein the implantable medical device is configured to allow an access of the external device to the implantable medical device (e.g. to control the implantable medical device) in case the provided authentication information satisfies a pre-defined criterion (e.g. authenticates the user as an authorized user).

Particularly, when the IMD is in the activated mode, the external device is configured to prompt the user to input said authentication information.

Further, according to an embodiment of the medical system, the external device is configured to control the implantable medical device when the external device has access to the implantable medical device.

Furthermore, according to an embodiment of the medical system, said authentication information comprises biometric data of the user.

Furthermore, in an embodiment of the medical system, said biometric data is one of: a heart rate of the user, a heart interval pattern of the user, a temperature of the user, a retina pattern of the user, a fingerprint of the user, a respiration rate of the user, a knuckle pattern of the user.

Furthermore, according to an embodiment of the medical system, the IMD and the external device are configured to measure biometric data of the user, wherein the external device is configured to transmit the measured biometric data measured by the external device from the external device to the IMD. Furthermore, in an embodiment of the medical system, the IMD is configured to permit access of the external device to the IMD if the transmitted biometric data matches the biometric data measured by the IMD. Particularly, the biometric data can be a series of heart intervals of the patient. Other biometric data of the patient (e.g. as disclosed herein) can also be used.

Particularly, according to an embodiment of the medical system, the external device is configured to request the user (e.g. a patient carrying the IMD implanted in the patient) to modify a respiration rate of the user (e.g. take three slow breaths), wherein the IMD is configured to measure the respiration rate of the user by means of the IMD. Particularly, in an embodiment, the IMD is configured to permit access of the external device to the IMD if the measured respiration rate matches the requested modification.

Furthermore, according to an embodiment of the medical system, when the IMD is in the activated mode the external device is configured to scan authentication information (e.g. a barcode) provided by the user and to compare the scanned authentication information with authentication information of the user stored in the IMD. Furthermore, in an embodiment, the IMD is configured to permit access of the external device to the IMD if the scanned authentication information corresponds to the authentication information stored in the IMD.

Furthermore, according to an embodiment of the medical system, when the IMD is in the activated mode, the external device is configured to prompt the user (e.g. a patient carrying the IMD implanted in the patient) to input authentication information (e.g. via the external device), wherein according to an embodiment said authentication information (e.g. one or several of: name, date of birth, address, Physician's Name, password, PIN) has been programmed into the IMD after implantation by means of a privileged external device (e.g. a programmer).

Particularly, according to an embodiment of the medical system, when the IMD is in the activated mode, the external device is configured to receive a password by the user (e.g. a patient carrying the IMD implanted in the patient). Particularly, in an embodiment, the IMD is configured to permit access of the external device to the IMD if the password input by the user matches a password stored in the 1MB.

Further, in an embodiment of the medical system, the external device and the IMD are configured to establish an encrypted connection between the external device and the IMD when the IMD is in the activated mode.

Further, in an embodiment of the medical system, the external device is configured to prompt the user through the external device to input the password that has been previously stored in the IMD.

Further, in an embodiment of the medical system, the external device is configured to transmit a representation of the inputted password via the encrypted connection to the IMD.

Furthermore, according to an embodiment of the medical system, the IMD is configured to decrypt the transmitted password representation and compare the transmitted password representation with the representation stored in the IMD.

Particularly, in an embodiment of the medical system, the IMD is configured to permit access of the external device to the 1MB if the decrypted password representation matches the password representation stored in the IMD, and to allow the external device to control the IMD.

Furthermore, according to an embodiment of the medical system, when the IMD is in the activated mode, the external device is configured to prompt the user (e.g. a patient carrying the IMD implanted in the patient) to move according to a pre-defined movement pattern, and wherein the IMD is configured to detect said movement pattern with an accelerometer in the IMD. Particularly, in an embodiment, the IMD is configured to permit access of the external device to the IMD if the detected pattern matches the pre-defined movement pattern. According to an example, the external device is configured to prompt the user to tap the IMD a plurality of times (e.g. five times) with a pre-defined pause (e.g. one second) in between each two successive taps. Alternatively, the external device can be configured to prompt the user to sit motionless for a pre-defined amount of time (e.g. 10 seconds).

According to an embodiment, the IMD is configured to detect vibrations transmitted from an external device, e.g. by placing the external device over the implant and generating vibrations which are transferred to the implant via tissue. For example, the IMD may sense vibrations using an accelerometer. For example, the external device comprises a vibration motor for generating vibrations serving as authentication signals. Exemplary external devices are smart phones or tablet computers.

Furthermore, according to an embodiment of the medical system, when the IMD is in the activated mode, the external device is configured to prompt the user (e.g. a patient carrying the IMD implanted in the patient) to place a hand over the IMD, and wherein the IMD is configured to detect a presence of the hand over the IMD by way of capacitive sensing. Particularly, in an embodiment, a further step of the method corresponds to permitting access to the IMD if a detection signal generated by the IMD matches a pre-defined reference confirming said presence of the hand over the IMD.

Alternatively, according to an embodiment, when the IMD is in the activated mode, the external device is configured to prompt the user (e.g. a patient carrying the IMD implanted in the patient) to press against the IMD, wherein the IMD is configured to detect a deformation of the IMD due to said pressing by means of a strain gauge comprises by the IMD. Particularly, in an embodiment, the IMD is configured to permit access of the external device to the IMD if a detection signal generated by the strain gauge matches a pre-defined reference confirming said pressing against the IMD.

Furthermore, according to an embodiment of the medical system, when the IMD is in the activated mode, the external device is configured to prompt the user (e.g. a patient carrying the IMD implanted in the patient) to press a button on the external device or to apply a magnetic field to the IMD for a second time.

Further, in an embodiment of the medical device, the IMD comprises a battery which is configured to be charged by a charging device of the medical system. Particularly, in an embodiment, the IMD is configured to permit access of the external device to the IMD if the IMD is in the activated mode and the battery is being charged by the charging device.

Further, in an embodiment of the medical system, when the IMD is in the activated mode, the external device or a further device of the system is configured to emit a light pattern, and wherein the IMD is configured to detect said light pattern by means of a light sensor of the IMD. Particularly, in an embodiment, the IMD is configured to permit access of the external device to the IMD if the detected light pattern corresponds to a pre-defined reference.

According to an embodiment of the present invention, an IMD is configured to be accessible authorized users via said authentication methods. Moreover, according to an embodiment, the IMD is configured to be set into a ‘safe mode’, which is a mode where enhanced safety measures are applied. For example, the safe mode could be accessible also users who are no authorized users. The IMD could provide an operational mode for authorized users and a mode for users without authorization.

Moreover, according to an embedment, a method for establishing privileged access of an external device to an implantable medical device is described, comprising the steps of:
Allowing the implantable medical device to assume an activated mode by letting a user of the implantable medical device apply a near field signal to the implantable medical device, wherein in the activated mode the implantable medical device is enabled to receive authentication information for authenticating the user of the implantable medical device, and
Providing authentication information to the implantable medical device, when the latter is in the activated mode to establish said access.

According to an embodiment, the IMD is configured to allow access for an unauthorized external device to a ‘safe-mode’ by providing a communications channel that is limited to performing that function. Compared to the activated mode, the ‘safe mode’ requires different, less or no authentication information need to be transferred from the external device to the IMD.

According to an embodiment of the present invention, the IMD, once entering the activated mode, starts a timer which expires after a predetermined time. The IMD is configured to deactivate the activated mode upon said expiration, and e.g. return to the previous operation mode.

In each of the above-described embodiments, access may only be permitted if in addition one or several further authentication procedures have also been completed successfully.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

In the following embodiments, further features and advantages of the present invention shall be described with reference to the Figure, wherein

FIG. 1 shows a schematic illustration of an embodiment of a medical system according to the present invention that can be used to conduct the method according to the present invention;

FIG. 2 shows a block diagram of embodiments of the method according to the present invention; and

FIG. 3 shows a block diagram corresponding to further embodiment of the method according to the present invention.

### DETAILED DESCIPTION

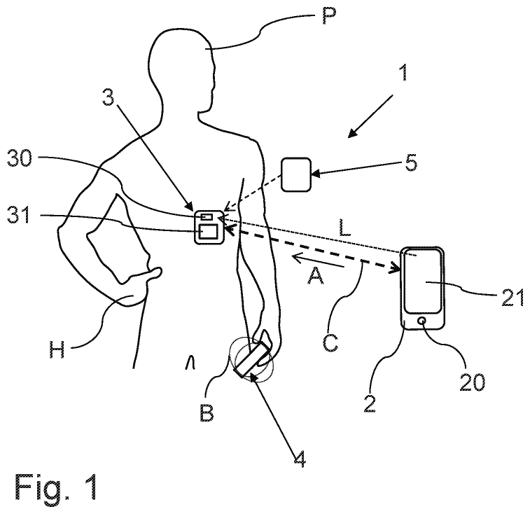

FIG. 1 shows an embodiment of a medical system 1 according to the present invention. According thereto, the medical system 1, comprises an implantable medical device (IMD) 3 (e.g. an implantable pacemaker, an implantable monitoring device, an implantable neurostimulator, etc., any implantable medical device which is capable of wireless communication with an external device or external data center), an external device 2, which can be any external device which is capable of wireless communication with an implantable medical device or a mobile device, such as a remote control or a smart phone, configured to control the implantable medical device 3 when the external device 2 has access to the implantable medical device 3 via a wireless connection C, and a near field communication device 4 configured to be manually positioned by a user P (e.g. a patient having the IMD implanted) of the implantable medical device 3 for applying a near field signal B to the implantable medical device 3, wherein the implantable medical device 3 is configured to assume an activated mode when the near field signal B is applied to the implantable medical device 3 by the near field communication device 4, and wherein in the activated mode the implantable medical device 3 is configured to receive authentication information A relating to the user P, and wherein the implantable medical device 3 is configured to allow an access of the external device 2 to the implantable medical device 3 in case the provided authentication information A satisfies a pre-defined criterion. Examples of such criteria will be described below. According to an embodiment, The near field communication device could be the same as the mobile/external device (2). For example, one could use the near field communications signals built into many mobile phones today.

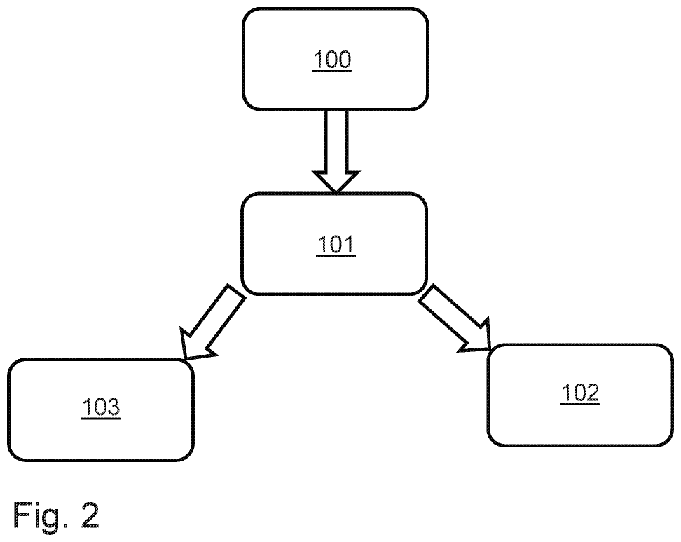

Thus, particularly, before the IMD 3 accepts a protected communication request (e.g., changing a program or requesting sensitive information) from the external device 2, the patient P must show intent to communicate. As an example, as shown in FIG. 2, the patient P can in a first step 100 place said near field communication device 4 over the IMD 3. The IMD 3 then detects the presence of the near field signal 4. Secondly, in further step 101 when initiating the communication request, the external device 2 can request the user P to provide authentication information in form of e.g. biometric data, for example to breathe at a certain rate for a given period of time (by using visual and/or haptic guidance) and the IMD 3 then measures the biometric data or compares the external device-measured biometric data to a stored value. Once the IMD 3 verifies the presence of the near field device and the validity of the biometric data, the IMD 3 accepts the communication request from the external device 102. Otherwise the IMD rejects the request for access 103.

Particularly, by requiring both physical access to the patient P/IMD 3 and customized information known only to the IMD 3 and the patient P to initiate communication, an actor that did not have both physical proximity and the customized information would be denied access.

Moreover, according to an embodiment of the present invention, the near field communication device is a magnet, wherein its magnetic field can be detected by the IMD.

Moreover, according to an embodiment of the present invention, the near field communication device is an NFC (Near Field Communication) protocol (similar to that used in contactless payment systems or keycards) that can be detected by the IMD.

According to a preferred embodiment, the IMD 3 is designed and configured to detect two or more authentication mechanisms (see list of potential authentication mechanisms below). Preferably, these mechanisms must be positively identified by the IMD 3 before allowing an external device 2 access to sensitive communication of the device 3.

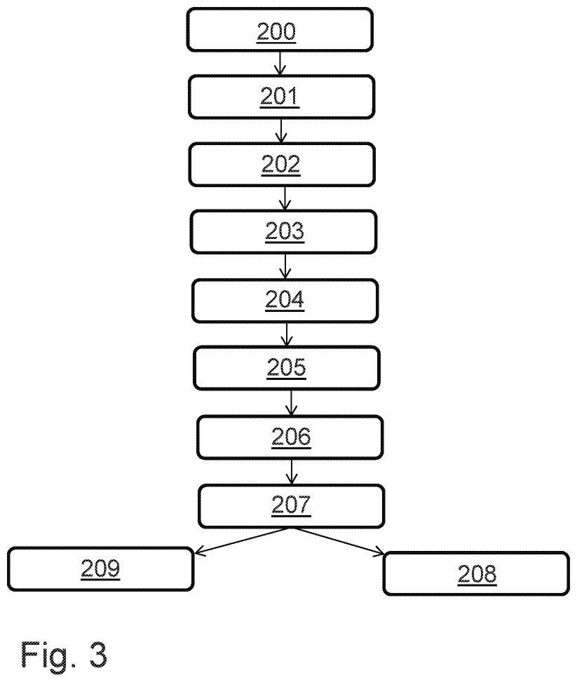

Particularly, according to an embodiment shown in FIG. 3, the required authentication information can be a password. Here, a possible process for handling multifactor authentication can be conducted as follows.

The implantable medical device (IMD) 3 is preferably provisioned at the factory with a standard firmware in a first step 200. No password or patient (P) specific details are present in the IMD.

In a further step 201, after implantation of the IMD 3 into the user patient P (wherein the implantation does not form part of the method according to the present invention), while visiting with a clinician, the user P provides a user specific password particularly forming a unique ID.

In a further step 202, while the clinician is adjusting the IMD 3 for the user P (using e.g. a device with elevated privileges), the clinician assigns the IMD 3 to the user P and programs the user's P password into the IMD 3.

In a further step 203, after the clinician's session ends, the user P will want to connect their external device (e.g. personal patient remote control device) to the IMD 3. Therefore, the user P first starts by applying the near field signal 4 (c.f. FIG. 1) to the IMD 3 for a specified time duration. This can be considered as a first factor of the multifactor scheme according to the present invention. Particularly, the near field communication device 4 provides a physical and proximity based interlock that reliably shows the user's P intent to connect a new device, namely external device 2 to the IMD 3.

In response, in succeeding step 204, the IMD 3 enters an activated mode that allows new devices to be connected to the IMD 3. Note that during normal communication modes, new devices cannot be added. Only previously added devices can establish a communication channel C (cf. FIG. 1).

In a further step 205, IMD 3 and the external device 2 (e.g. patient remote) establish preliminary security using encryption.

Once a preliminary connection is established, a user interface 21 of the external device 2 prompts the user P in step 206 for the password that had been previously programmed into the implant during the clinician's session in step 202.

In succeeding step 206, the password A (cf. FIG. 1) is inputted by the user P and the password representation (e.g., a cryptographic hash) is transmitted to the IMD 3 via the encrypted (secure) communications channel C.

In response, in step 207, the IMD 3 decrypts the transmitted password representation and compares it to its internal representation.

If the password representation A matches, then the user P is authenticated and the new external device 2 (e.g. patient remote control device) is added (or paired) to the IMD 3 (208). If the password representation A does not match, then the external device 2 is not allowed to control the IMD 3 (209).

Note that other permutations of this approach are also possible. For example, a unique password (per IMD 3) can be programmed at the factory and printed on a card that is packed with the IMD 3. To make the process even more convenient, the unique password can be encoded as a QR code and the information can be imported with a camera. When the clinician sets up the IMD 3 for the first time, this password would be required to connect to the clinician's programmer. This makes the system 1 more secure, since there would be no channel to the IMD 3 that requires only a single factor.

As further illustrated in FIG. 2 in conjunction with FIG. 1, instead of password also other authentication information can be used in the present invention.

As already mentioned above, biometric data such as heart rate, heart interval pattern, temperature, retina pattern, fingerprint, respiration rate, knuckle pattern of the user P can be used to verify patient authenticity.

For example, after bringing the IMD to its activated mode in step 100, both the IMD 3 and the external device 2 could measure a series of heart intervals, the external device 2 could then transmit the intervals to the IMD 3 via connection C (101). The IMD 3 then only permits access 102 if the transmitted interval series matches the IMD measured interval series (optionally along with one or more other authentication mechanism). Otherwise, the IMD 3 rejects access 103.

Furthermore, according to an alternative example, the external device 2 could ask the user P in step 101 to modify their respiration rate (e.g., take 3 slow breaths) and the IMD 3 could measure the respiration rate. The IMD 3 then only permits access 102 if the respiration rate decreases for (at least) 3 breaths (optionally along with one or more other authentication mechanism). Otherwise, the IMD 3 rejects access of the external device to the IMD (103).

According to a further embodiment, after bringing the IMD to its activated mode in step 100 using a near field communication device 4 (cf. FIG. 1), the user 4 scans a barcode or inputs authentication information using the external device 2 in step 101, which authentication information was generated for the IMD 3 at manufacturing time to verify that the patient P is the one initiating security (optionally along with one or more other authentication mechanism). The IMD 3 then only permits access 102 if the authentication information provided by the user P matches the information stored in the IMD 3. Otherwise, the IMD 3 rejects access of the external device to the IMD (103).

Furthermore, according to yet another embodiment illustrated in FIGS. 1 and 2, the authentication information A (e.g. name, date of birth, address, attending physician, password, PIN, etc.) can be programmed into the IMD 3 just after implantation by a privileged external device (programmer). Normally these fields are not writable by a patient remote type device. During the security exchange 101, the external device 2 can provide this information (or a cryptographic hash) to complete access 102 (optionally along with one or more other authentication mechanism).

According to a further example illustrated in FIGS. 1 and 2, after application of the near field signal 4 to force the IMD 3 to enter the activated mode (100), the external device 2 can ask the user P to tap the IMD 3 with a defined pattern in step 101 or to sit still or move while initiating communication (101). The IMD 3 can then detect the tap pattern or movement using a built-in accelerometer 30. The IMD 3 then only permits access 102 if the tap pattern or movement matches its expectations (optionally along with one or more other authentication mechanism). Otherwise, the IMD 3 rejects the request of external device 2 to access/control IMD 3 (103).

According to a further example illustrated in FIGS. 1 and 2, after application of the near field communication device 4 to force the IMD 3 to enter the activated mode (100), the external device 2 can ask the user P to place their hand H over the IMD 3 or to press on the IMD 3 (101). The IMD 3 can then use capacitive sensing 30 to detect the presence of the hand H or a strain gauge 30 to sense flexing of the IMD 3 (101). Access would be granted (102) if capacitive and/or strain gauge measurements meet expectations (optionally along with one or more other authentication mechanism). Otherwise, the IMD 3 rejects the request of external device 2 to access/control IMD 3 (103).

According to a further example (cf. FIG. 1), while initiating communication, the patient P may also press a button 20 on the external device 2 (or apply said near field communication device 4) to confirm the patient P really is the one attempting to unlock security (optionally along with one or more other authentication mechanism). Note, that this may be used after communication initiation has already started and not as a trigger to start communication.

According to a further example illustrated in FIGS. 1 and 2, after application of the near field communication device 4 (100) and while establishing communications, the user P applies a charging device 5 to the IMD 3 in step 101 in order to charge a battery 31 of the IMD 3. The IMD 3 then only permits access (102) if the battery 31 is actually charging (optionally along with one or more other authentication mechanism). Otherwise, the IMD 3 rejects the request of external device 2 to access/control IMD 3 (103).

Finally, according to a further example, after application of the near field signal 4 (100) to trigger the IMD 3 to enter its activated mode, a light sensor 30 embedded in the IMD 3 can be used to receive pulses of light L from the external device 2 (or from a further device). Particularly such light pattern L may be generated with a camera flash LED). This could be a simple mechanism (on/off) or a way to encode small amounts of data.

Particularly, the system 1 and method according to the present invention provide increased security due to the requirement of multiple authentication factors before allowing protected communication access to the IMD 3. If properly implemented, attacks from remote unauthorized users would be minimized, increasing the level of cybersecurity while maintaining ease of use for the patient P. Additionally, the suggested mechanisms are simple, economical and easily accessible by the patient/user P while being difficult to access by an unauthorized user. Particularly, the possibility of using two or more authentication methods that do not involve having a display and/or keyboard on both devices 2, 3 makes the approach according to the present invention particularly valuable in the context of implantable medical device systems 1.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A method for establishing an access of an external device to an implantable medical device, comprising the steps of:
Allowing the implantable medical device to assume an activated mode by letting a user of the implantable medical device apply a near field signal to the implantable medical device, wherein in the activated mode the implantable medical device is enabled to receive authentication information for authenticating the user of the implantable medical device, and
Providing authentication information to the implantable medical device, when the latter is in the activated mode to establish said access.

2. The method according to claim 1, wherein said near field signal is applied by placing a near field communication device in proximity to the implantable medical device.

3. The method according to claim 2, wherein the near field communication device is a magnet.

4. The method according to claim 1, wherein the method further comprises allowing the external device to control the implantable medical device when the external device has access to the implantable medical device.

5. The method according to claim 1, wherein said authentication information comprises biometric data of the user.

6. The method according to claim 5, wherein said biometric data is one of: a heart rate of the user, a heart interval pattern of the user, a temperature of the user, a retina pattern of the user, a fingerprint of the user, a respiration rate of the user, a knuckle pattern of the user.

7. The method according to claim 1, wherein providing said authentication information comprises measuring biometric data of the user by means of the implantable medical device as well as by means of the external device, and transmitting the measured biometric data measured by the external device the external device to the implantable medical device.

8. The method according to claim 1, wherein providing said authentication information comprises requesting the user to modify a respiration rate of the user (-R)--and measuring the respiration rate of the user by means of the implantable medical device.

9. The method according to claim 1, wherein providing said authentication information to establish said access involves inputting authentication information by the user via the external device, which authentication information has been stored in the implantable medical device before, particularly during manufacturing of the implantable medical device.

10. The method according to claim 1, wherein providing said authentication information comprises inputting authentication information by the user via the external device, wherein particularly the authentication information has been programmed into the implantable medical device after implantation of the implantable medical device by means of a programming device.

11. The method according to claim 1, wherein providing said authentication information involves inputting of a password by the user via the external device.

12. The method according to claim 1, wherein providing said authentication information comprises prompting the user to move according to a pre-defined movement pattern, and detecting said movement pattern with an accelerometer contained in the implantable medical device.

13. The method according to claim 1, wherein providing said authentication information comprises prompting the user through the external device to place a hand over the implantable medical device, and detecting the presence of the hand by means of a capacitive sensor of the implantable medical device.

14. The method according to claim 1, wherein providing said authentication information comprising prompting the user through the external device to press against the implantable medical device, and detecting a deformation of the implantable medical device due to said pressing by means of a strain gauge of the implantable medical device.

15. The method according to claim 1, wherein providing said authentication information to establish said access involves prompting the user through the external device to press a button on the external device to send a message to the implant or to apply a near field signal to the implantable medical device for a second time.

16. The method according to claim 1, wherein providing said authentication information to establish said access comprises applying a charging device to the implantable medical device to charge a battery of the implantable medical device.

17. The method according to claim 1, wherein providing said authentication information to establish said access comprises emitting a light pattern, and detecting said light pattern by means of a light sensor of the implantable medical device.

18. A medical system, comprising:
an implantable medical device,
an external device configured to control the implantable medical device when the external device has access to the implantable medical device,
a near field communication device configured to be manually positioned by a user of the implantable medical device for applying a near field signal to the implantable medical device, wherein the implantable medical device is configured to assume an activated mode when the near field signal is applied to the implantable medical device by the near field communication device, and wherein in the activated mode the implantable medical device is configured to receive authentication information relating to the user, and wherein the implantable medical device is configured to allow an access of the external device to the implantable medical device in case the provided authentication information satisfies a pre-defined criterion.

19. The medical system according to claim 18, wherein the near field communication device is integrated in the external device.
